(19) United States
(12) Patent Application Publication
(10) Pub. No.: US 2025/0327130 A1
(43) Pub. Date: Oct. 23, 2025

(54) USE OF CELL-FREE DNA FRAGMENTOMES IN THE DIAGNOSTIC EVALUATION OF PATIENTS WITH SIGNS AND SYMPTOMS SUGGESTIVE OF CANCER

(71) Applicant: Delfi Diagnostics, Inc., Baltimore, MD (US)

(72) Inventors: Peter Bach, Baltimore, MD (US); Tara Maddala, Baltimore, MD (US); Alessandro Leal, Baltimore, MD (US); Debbie Jakubowski, Baltimore, MD (US); Tony Wu, Baltimore, MD (US)

(21) Appl. No.: 18/864,428

(22) PCT Filed: May 12, 2023
(86) PCT No.: PCT/US2023/022104

§ 371 Date: Nov. 8, 2024

Related U.S. Application Data
(60) Provisional application No. 63/341,323, filed on May 12, 2022.

(51) Int. Cl. C12Q 1/6886; C12N 15/10

(52) U.S. Cl. CPC C12Q 1/6886; C12N 15/1093; C12Q 2600/156

(57) ABSTRACT

The present disclosure provides methods of uses thereof for improved diagnostic applications using genome-wide patterns of fragmented cell-free DNA (cfDNA) from plasma, derived by low-coverage whole-genome sequencing, and analyzed in conjunction with certain clinical and demographic features of individual patients. In particular, the present invention provides new and effective methods for confirming the presence or absence of cancer in an individual patient already suspected of having cancer.

### CROSS REFERENCE TO RELATED APPLICATIONS

This application claims the benefit of priority under 35 U.S.C. § 119(e) of U.S. Provisional Patent Application Ser. No. 63/341,323 filed on May 12, 2022. The disclosure of the prior application is considered part of and is herein incorporated by reference in the disclosure of this application in its entirety.

### FIELD OF THE INVENTION

The present invention relates generally to the diagnosis of cancer and more specifically to the analysis of genome-wide patterns of fragmented cell-free DNA (cfDNA) in conjunction with clinical and demographic features of individual patients.

### BACKGROUND

Individuals may develop symptoms or have abnormal imaging results suggestive of but not definitive for certain types of cancer. For example, individuals, especially those with smoking histories, may develop symptoms or have abnormal chest imaging results suggestive of but not definitive for lung cancer. The diagnostic workup can be convoluted for these individuals, delaying diagnostic resolution. Delays to definitive diagnosis can cause anxiety in patients and delays to treatment, which can adversely affect clinical outcomes, especially for those who could benefit from surgery.

Reasons for the delay in diagnosis are diverse and often related to the nonspecific nature of lung cancer symptoms. Patients with late-stage lung cancer may present with severe symptoms that lead quickly to a definitive diagnosis, but those with earlier-stage lung cancer may have general symptoms (e.g., aches and pains, cough, fatigue, shortness of breath, and weight loss) that do not alarm patients enough to seek medical attention or can be mistaken easily for a chronic or benign condition. Consequently, patients may undergo unnecessary treatments before being evaluated for lung cancer, thereby prolonging the time to diagnostic resolution. While these reasons may affect any given patient, the often-convoluted path to diagnosis may be particularly detrimental for groups that already face suboptimal lung cancer outcomes based on race/ethnicity, socioeconomic status, and/or sex.

Recognizing the need to improve the diagnostic pathway for lung cancer, medical societies worldwide have developed guidelines for the diagnostic workup of patients with nonspecific respiratory symptoms or suspected lung cancer (Table 3). Although the guidelines agree to some extent on what signs and symptoms should trigger follow-up procedures (e.g., chest pain, cough, fatigue, finger clubbing, hemoptysis, shortness of breath, and weight loss), they differ in the specific triggering signs/symptoms, the patient type the signs/symptoms arise in, and what follow-up should occur. These variations in the guidelines likely reflect the limited value of these symptoms as predictors of lung cancer, alone or in combination.

### SUMMARY OF THE INVENTION

The present invention is based on the seminal discovery that the characterizing genome-wide patterns of fragmentation of cell-free DNA (cfDNA) in plasma using low-coverage whole-genome sequencing can improve cancer diagnosis when analyzed in conjunction with certain clinical and demographic features of individual patients.

In one embodiment, the present invention provides methods for processing cfDNA fragments from a sample obtained from a subject and generating sequencing libraries; subjecting the sequencing libraries to whole genome sequencing to obtain sequenced fragments, wherein genome coverage is about 9× to 0.1×; mapping the sequenced fragments to a genome to obtain genomic intervals of mapped sequences; analyzing the genomic intervals of mapped sequences to determine cfDNA fragment lengths and amounts to establish a composite cfDNA fragmentation profile using the cfDNA fragment lengths and amounts; analyzing one or more demographic or clinical characteristics from the subject which are associated with a type of cancer to be identified; and detecting a composite cfDNA fragmentation profile based on lengths and amounts that is variable relative to a reference cfDNA fragmentation profile from a healthy subject, wherein increased variability of the cfDNA fragmentation profile and the presence of one or more demographic or clinical characteristics indicate that the subject has the type of cancer.

In some aspects, the genomic intervals of mapped sequences are non-overlapping. In certain aspects, the genomic intervals each include thousands to millions of base pairs.

In further aspects, a cfDNA fragmentation profile is determined within each genomic intervals. In some such aspects, the cfDNA fragmentation profile includes a median fragment size. In further aspects, the cfDNA fragmentation profile includes a fragment size distribution.

In additional aspects, the type of cancer to be identified is selected from the group consisting of head and neck cancer, lung cancer, breast cancer, esophageal cancer, gastric cancer, bile duct cancer, liver cancer, pancreatic cancer, colorectal cancer, kidney cancer, bladder cancer, ovarian cancer, and endometrial cancer. In certain such aspects, the type of cancer to be identified is lung cancer.

In more aspects, the one or more clinical characteristic is selected from the group consisting of pain, involuntary weight loss, fever, fatigue, skin changes, dyspnea, cough, hoarseness, dysphagia, unusual bleeding, anemia, change in intestinal or urinary habits, or swelling or lumps anywhere in the subject's body.

In more aspects, the one or more demographic characteristic is selected from the group consisting of age, sex and smoking status.

In further aspects, a subject identified as having a type of cancer is administered a therapeutic agent suitable for the treatment of the type of cancer.

### BRIEF DESCRIPTION OF THE DRAWINGS

FIG. 1 illustrates that median DELFI scores are higher for individuals with lung cancer than those with no cancer, for any given symptom reported

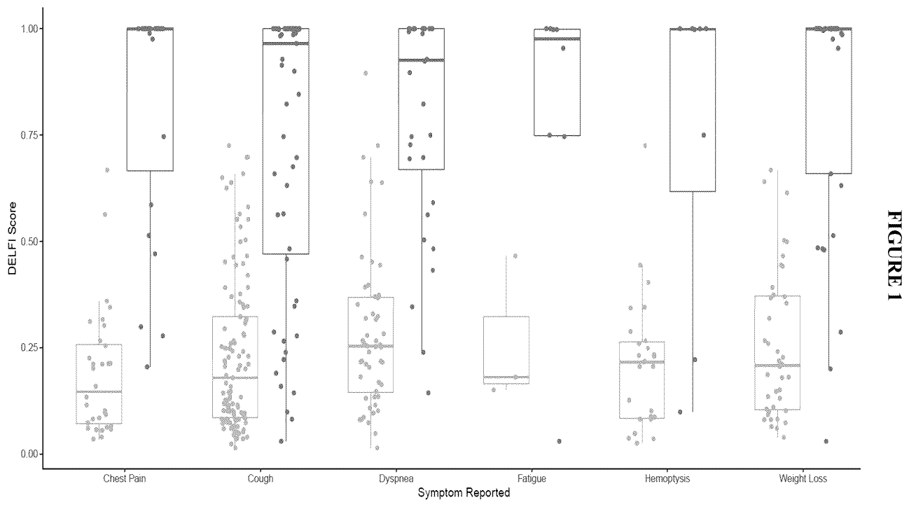

FIG. 2 illustrates that median DELFI scores are higher for individuals with lung cancer than those with no cancer, for number of symptoms reported.

FIG. 3 illustrates that the methods disclosed using the DELFI score predicts lung cancer better than symptoms alone.

FIG. 4 illustrates an exemplary embodiment of the disclosed workflow.

FIG. 5 shows a comparison of cfDNA fragmentation profiles in patients with no lung cancer, no lung cancer but with benign nodules, and lung cancer illustrating that DELFI discriminates lung cancer vs no cancer even in a symptomatic cohort, suggesting clinical utility to stream the diagnostic work up for this patient population.

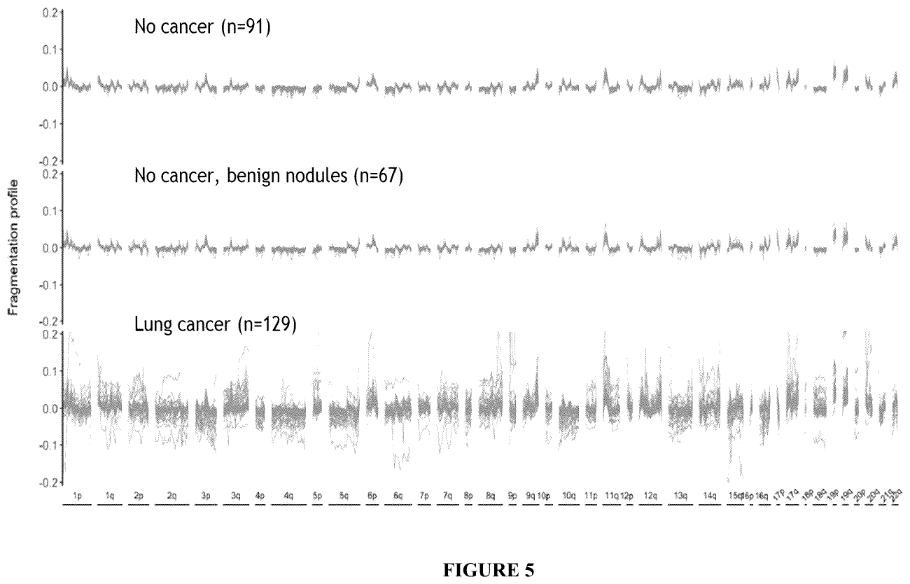

FIG. 6 is an example computer 800 that may be used to implement the methods described herein.

### DETAILED DESCRIPTION

The present invention is based on the seminal discovery that the characterizing genome-wide patterns of fragmentation of cell-free DNA (cfDNA) in plasma using low-coverage whole-genome sequencing improves cancer diagnosis when analyzed in conjunction with certain clinical and demographic features of individual patients.

Described herein are methods for processing cfDNA fragments from a sample obtained from a subject and generating sequencing libraries; subjecting the sequencing libraries to whole genome sequencing to obtain sequenced fragments, wherein genome coverage is about 9× to 0.1×; mapping the sequenced fragments to a genome to obtain genomic intervals of mapped sequences; analyzing the genomic intervals of mapped sequences to determine cfDNA fragment lengths and amounts to establish a composite cfDNA fragmentation profile using the cfDNA fragment lengths and amounts; analyzing one or more demographic or clinical characteristics from the subject which are associated with a type of cancer to be identified; and detecting a composite cfDNA fragmentation profile based on lengths and amounts that is variable relative to a reference cfDNA fragmentation profile from a healthy subject, wherein increased variability of the cfDNA fragmentation profile and the presence of one or more demographic or clinical characteristics indicate that the subject has the type of cancer.

In some aspects, the genomic intervals of mapped sequences are non-overlapping. In certain aspects, the genomic intervals each include thousands to millions of base pairs.

In further aspects, a cfDNA fragmentation profile is determined within each genomic intervals. In some such aspects, the cfDNA fragmentation profile includes a median fragment size. In further aspects, the cfDNA fragmentation profile includes a fragment size distribution.

In additional aspects, the type of cancer to be identified is selected from the group consisting of head and neck cancer, lung cancer, breast cancer, esophageal cancer, gastric cancer, bile duct cancer, liver cancer, pancreatic cancer, colorectal cancer, kidney cancer, bladder cancer, ovarian cancer, and endometrial cancer. In certain such aspects, the type of cancer to be identified is lung cancer.

In more aspects, the one or more clinical characteristic is selected from the group consisting of pain, involuntary weight loss, fever, fatigue, skin changes, dyspnea, cough, hoarseness, dysphagia, unusual bleeding, anemia, change in intestinal or urinary habits, or swelling or lumps anywhere in the subject's body.

In more aspects, the one or more demographic characteristic is selected from the group consisting of age, sex and smoking status.

In further aspects, a subject identified as having a type of cancer is administered a therapeutic agent suitable for the treatment of the type of cancer.

Before the present compositions and methods are described, it is to be understood that this invention is not limited to the particular methods and systems described, as such methods and systems may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

### Example Hardware Implementation

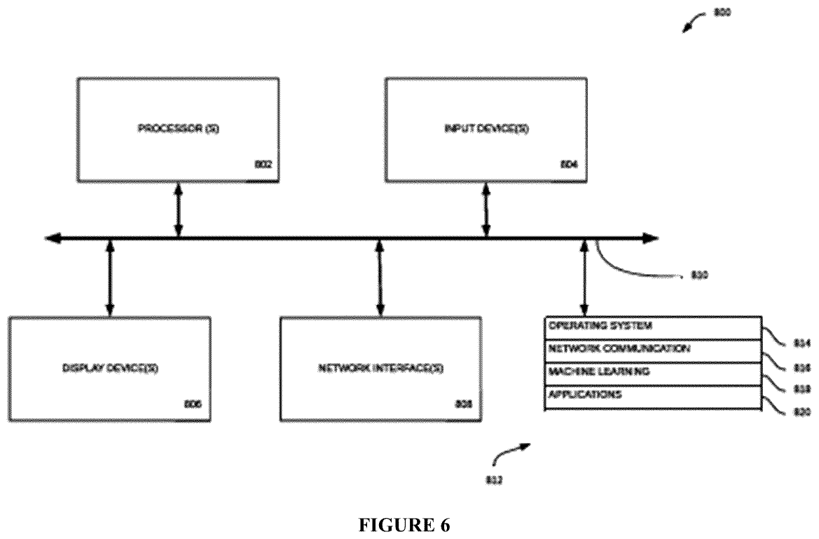

FIG. 6 illustrates an example computer 800 that may be used to implement the methods described herein. For example, the computer 800 may include a machine learning system that trains a machine learning model to generate a cfDNA fragmentation profile, a cancer diagnosis or any combination thereof as described above or a portion or combination thereof in some embodiments. The computer 800 may be any electronic device that runs software applications derived from compiled instructions, including without limitation personal computers, servers, smart phones, media players, electronic tablets, game consoles, email devices, etc. In some implementations, the computer 800 may include one or more processors 802, one or more input devices 804, one or more display devices 806, one or more network interfaces 808, and one or more computer-readable mediums 812. Each of these components may be coupled by bus 810, and in some embodiments, these components may be distributed among multiple physical locations and coupled by a network.

Display device 806 may be any known display technology, including but not limited to display devices using Liquid Crystal Display (LCD) or Light Emitting Diode (LED) technology. Processor(s) 802 may use any known processor technology, including but not limited to graphics processors and multi-core processors. Input device 804 may be any known input device technology, including but not limited to a keyboard (including a virtual keyboard), mouse, track ball, camera, and touch-sensitive pad or display. Bus 810 may be any known internal or external bus technology, including but not limited to ISA, EISA, PCI, PCI Express, USB, Serial ATA or FireWire. Computer-readable medium 812 may be any non-transitory medium that participates in providing instructions to processor(s) 804 for execution, including without limitation, non-volatile storage media (e.g., optical disks, magnetic disks, flash drives, etc.), or volatile media (e.g., SDRAM, ROM, etc.).

Computer-readable medium 812 may include various instructions 814 for implementing an operating system (e.g., Mac OS®, Windows®, Linux). The operating system may be multi-user, multiprocessing, multitasking, multithreading, real-time, and the like. The operating system may perform basic tasks, including but not limited to: recognizing input from input device 804; sending output to display device 806; keeping track of files and directories on computer-readable medium 812; controlling peripheral devices (e.g., disk drives, printers, etc.) which can be controlled directly or through an I/O controller; and managing traffic on bus 810. Network communications instructions 816 may establish and maintain network connections (e.g., software for implementing communication protocols, such as TCP/IP, HTTP, Ethernet, telephony, etc.).

Machine learning instructions 818 may include instructions that enable computer 800 to function as a machine learning system and/or to training machine learning models to generate DMS values as described herein. Application(s) 820 may be an application that uses or implements the processes described herein and/or other processes. The processes may also be implemented in operating system 814. For example, application 820 and/or operating system may create tasks in applications as described herein.

The described features may be implemented in one or more computer programs that may be executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program may be written in any form of programming language (e.g., Objective-C, Java), including compiled or interpreted languages, and it may be deployed in any form, including as a stand-alone program or as a module, component, subroutine, or other unit suitable for use in a computing environment.

Suitable processors for the execution of a program of instructions may include, by way of example, both general and special purpose microprocessors, and the sole processor or one of multiple processors or cores, of any kind of computer. Generally, a processor may receive instructions and data from a read-only memory or a random-access memory or both. The essential elements of a computer may include a processor for executing instructions and one or more memories for storing instructions and data. Generally, a computer may also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data may include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory may be supplemented by, or incorporated in, ASICs (application-specific integrated circuits).

To provide for interaction with a user, the features may be implemented on a computer having a display device such as an LED or LCD monitor for displaying information to the user and a keyboard and a pointing device such as a mouse or a trackball by which the user can provide input to the computer.

The features may be implemented in a computer system that includes a back-end component, such as a data server, or that includes a middleware component, such as an application server or an Internet server, or that includes a front-end component, such as a client computer having a graphical user interface or an Internet browser, or any combination thereof. The components of the system may be connected by any form or medium of digital data communication such as a communication network. Examples of communication networks include, e.g., a telephone network, a LAN, a WAN, and the computers and networks forming the Internet.

The computer system may include clients and servers. A client and server may generally be remote from each other and may typically interact through a network. The relationship of client and server may arise by virtue of computer programs running on the respective computers and having a client-server relationship to each other.

One or more features or steps of the disclosed embodiments may be implemented using an Application Programming Interface (API). An API may define one or more parameters that are passed between a calling application and other software code (e.g., an operating system, library routine, function) that provides a service, that provides data, or that performs an operation or a computation.

The API may be implemented as one or more calls in program code that send or receive one or more parameters through a parameter list or other structure based on a call convention defined in an API specification document. A parameter may be a constant, a key, a data structure, an object, an object class, a variable, a data type, a pointer, an array, a list, or another call. API calls and parameters may be implemented in any programming language. The programming language may define the vocabulary and calling convention that a programmer will employ to access functions supporting the API.

In some implementations, an API call may report to an application the capabilities of a device running the application, such as input capability, output capability, processing capability, power capability, communications capability, etc.

While various embodiments have been described above, it should be understood that they have been presented by way of example and not limitation. It will be apparent to persons skilled in the relevant art(s) that various changes in form and detail can be made therein without departing from the spirit and scope. In fact, after reading the above description, it will be apparent to one skilled in the relevant art(s) how to implement alternative embodiments. For example, other steps may be provided, or steps may be eliminated, from the described flows, and other components may be added to, or removed from, the described systems. Accordingly, other implementations are within the scope of the following claims.

In addition, it should be understood that any figures which highlight the functionality and advantages are presented for example purposes only. The disclosed methodology and system are each sufficiently flexible and configurable such that they may be utilized in ways other than that shown.

Although the term “at least one” may often be used in the specification, claims and drawings, the terms “a”, “an”, “the”, “said”, etc. also signify “at least one” or “the at least one” in the specification, claims and drawings.

Finally, it is the applicant's intent that only claims that include the express language “means for” or “step for” be interpreted under 35 U.S.C. 112(f). Claims that do not expressly include the phrase “means for” or “step for” are not to be interpreted under 35 U.S.C. 112(f).

The presently described methods and systems are useful for detecting, cancer in a subject and optionally treating the cancer subject. Any appropriate subject, such as a mammal can be assessed, and/or treated as described herein. Examples of some mammals that can be assessed, and/or treated as described herein include, without limitation, humans, primates such as monkeys, dogs, cats, horses, cows, pigs, sheep, mice, and rats. For example, a human having, or suspected of having, cancer can be assessed using a method described herein and, optionally, can be treated with one or more cancer treatments as described herein. The methods disclosed herein may include administering to the subject identified as having the type of cancer, a therapeutic agent suitable for the treatment of the type of cancer.

A subject having, or suspected of having, any appropriate type of cancer can be assessed, and/or treated (e.g., by administering one or more cancer treatments to the subject) using the methods and systems described herein. A cancer can be any stage cancer. In some aspects, a cancer can be an early stage cancer. In some aspects, a cancer can be an asymptomatic cancer. In some aspects, a cancer can be a residual disease and/or a recurrence (e.g., after surgical resection and/or after cancer therapy). A cancer can be any type of cancer. Examples of types of cancers that can be assessed, and/or treated as described herein include, without limitation, lung, colorectal, prostate, breast, pancreas, bile duct, liver, CNS, stomach, esophagus, gastrointestinal stromal tumor (GIST), uterus and ovarian cancer. Additional types of cancers include, without limitation, myeloma, multiple myeloma, B-cell lymphoma, follicular lymphoma, lymphocytic leukemia, leukemia and myelogenous leukemia. In some aspects, the cancer is a solid tumor. In some aspects, the cancer is a sarcoma, carcinoma, or lymphoma. In some aspects, the cancer is lung, colorectal, prostate, breast, pancreas, bile duct, liver, CNS, stomach, esophagus, gastrointestinal stromal tumor (GIST), uterus or ovarian cancer. In some aspects, the cancer is a hematologic cancer. In some aspects, the cancer is myeloma, multiple myeloma, B-cell lymphoma, follicular lymphoma, lymphocytic leukemia, leukemia or myelogenous leukemia.

When treating a subject having, or suspected of having, cancer as described herein, the subject can be administered one or more cancer treatments. A cancer treatment can be any appropriate cancer treatment. One or more cancer treatments described herein can be administered to a subject at any appropriate frequency (e.g., once or multiple times over a period of time ranging from days to weeks). Examples of cancer treatments include, without limitation, surgical intervention, adjuvant chemotherapy, neoadjuvant chemotherapy, radiation therapy, hormone therapy, cytotoxic therapy, immunotherapy, adoptive T cell therapy (e.g., chimeric antigen receptors and/or T cells having wild-type or modified T cell receptors), targeted therapy such as administration of kinase inhibitors (e.g., kinase inhibitors that target a particular genetic lesion, such as a translocation or mutation), (e.g., a kinase inhibitor, an antibody, a bispecific antibody), signal transduction inhibitors, bispecific antibodies or antibody fragments (e.g., BiTEs), monoclonal antibodies, immune checkpoint inhibitors, surgery (e.g., surgical resection), or any combination of the above. In some aspects, a cancer treatment can reduce the severity of the cancer, reduce a symptom of the cancer, and/or to reduce the number of cancer cells present within the subject.

In some aspects, a cancer treatment can be a chemotherapeutic agent. Non-limiting examples of chemotherapeutic agents include: amsacrine, azacitidine, axathioprine, bevacizumab (or an antigen-binding fragment thereof), bleomycin, busulfan, carboplatin, capecitabine, chlorambucil, cisplatin, cyclophosphamide, cytarabine, dacarbazine, daunorubicin, docetaxel, doxifluridine, doxorubicin, epirubicin, erlotinib hydrochlorides, etoposide, fiudarabine, floxuridine, fludarabine, fluorouracil, gemcitabine, hydroxyurea, idarubicin, ifosfamide, irinotecan, lomustine, mechlorethamine, melphalan, mercaptopurine, methotrxate, mitomycin, mitoxantrone, oxaliplatin, paclitaxel, pemetrexed, procarbazine, all-trans retinoic acid, streptozocin, tafluposide, temozolomide, teniposide, tioguanine, topotecan, uramustine, valrubicin, vinblastine, vincristine, vindesine, vinorelbine, and combinations thereof. Additional examples of anti-cancer therapies are known in the art; see, e.g., the guidelines for therapy from the American Society of Clinical Oncology (ASCO), European Society for Medical Oncology (ESMO), or National Comprehensive Cancer Network (NCCN).

In one embodiment, the methods and systems described herein may also be used in monitoring a subject having, or suspected of having, cancer as described herein. In some aspects the monitoring can be before, during, and/or after the course of a cancer treatment. Methods of monitoring provided herein can be used to determine the efficacy of one or more cancer treatments and/or to select a subject for increased monitoring.

In some aspects, the monitoring can include conventional techniques capable of monitoring one or more cancer treatments (e.g., the efficacy of one or more cancer treatments). In some aspects, a subject selected for increased monitoring can be administered a diagnostic test (e.g., any of the diagnostic tests disclosed herein) at an increased frequency compared to a subject that has not been selected for increased monitoring. For example, a subject selected for increased monitoring can be administered a diagnostic test at a frequency of twice daily, daily, bi-weekly, weekly, bi-monthly, monthly, quarterly, semi-annually, annually, or any at frequency therein.

In various aspects, DNA is present in a biological sample taken from a subject and used in the methodology of the invention. The biological sample can be virtually any type of biological sample that includes DNA. The biological sample is typically a fluid, such as whole blood or a portion thereof with circulating cfDNA. In embodiments, the sample includes DNA from a tumor or a liquid biopsy, such as, but not limited to amniotic fluid, aqueous humor, vitreous humor, blood, whole blood, fractionated blood, plasma, serum, breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensates, sebum, semen, sputum, sweat, synovial fluid, tears, vomit, prostatic fluid, nipple aspirate fluid, lachrymal fluid, perspiration, cheek swabs, cell lysate, gastrointestinal fluid, biopsy tissue and urine or other biological fluid. In one aspect, the sample includes DNA from a circulating tumor cell.

As disclosed above, the biological sample can be a blood sample. The blood sample can be obtained using methods known in the art, such as finger prick or phlebotomy. Suitably, the blood sample is approximately 0.1 to 20 ml, or alternatively approximately 1 to 15 ml with the volume of blood being approximately 10 ml. Smaller amounts may also be used, as well as circulating free DNA in blood. Microsampling and sampling by needle biopsy, catheter, excretion or production of bodily fluids containing DNA are also potential biological sample sources.

The methods and systems of the disclosure utilize nucleic acid sequence information and can therefore include any method or sequencing device for performing nucleic acid sequencing including nucleic acid amplification, polymerase chain reaction (PCR), nanopore sequencing, 454 sequencing, insertion tagged sequencing. In some aspects, the methodology or systems of the disclosure utilize systems such as those provided by Illumina, Inc, (including but not limited to HiSeq™ X10, HiSeq™ 1000, HiSeq™ 2000, HiSeq™ 2500, Genome Analyzers™, MiSeq™. NextSeq, NovaSeq 6000 systems), Applied Biosystems Life Technologies (SOLiD™ System, Ion PGM™ Sequencer, ion Proton™ Sequencer) or Genapsys or BGI MGI and other systems. Nucleic acid analysis can also be carried out by systems provided by Oxford Nanopore Technologies (GridiON™, MiniON™) or Pacific Biosciences (Pacbio™ RS II or Sequel I or II).

The present invention includes systems for performing steps of the disclosed methods and is described partly in terms of functional components and various processing steps. Such functional components and processing steps may be realized by any number of components, operations and techniques configured to perform the specified functions and achieve the various results. For example, the present invention may employ various biological samples, biomarkers, elements, materials, computers, data sources, storage systems and media, information gathering techniques and processes, data processing criteria, statistical analyses, regression analyses and the like, which may carry out a variety of functions.

Accordingly, the invention further provides a system for detecting, analyzing, and/or assessing cancer. In various aspects, the system includes: (a) a sequencer configured to generate a low-coverage whole genome sequencing data set for a sample; and (b) a computer system and/or processor with functionality to perform a method of the invention.

In some aspects, the computer system further includes one or more additional modules. For example, the system may include one or more of an extraction and/or isolation unit operable to select suitable genetic components analysis, e.g., cfDNA fragments of a particular size.

In some aspects, the computer system further includes a visual display device. The visual display device may be operable to display a curve fit line, a reference curve fit line, and/or a comparison of both.

Methods for detection and analysis according to various aspects of the present invention may be implemented in any suitable manner, for example using a computer program operating on the computer system. As discussed herein, an exemplary system, according to various aspects of the present invention, may be implemented in conjunction with a computer system, for example a conventional computer system comprising a processor and a random access memory, such as a remotely-accessible application server, network server, personal computer or workstation. The computer system also suitably includes additional memory devices or information storage systems, such as a mass storage system and a user interface, for example a conventional monitor, keyboard and tracking device. The computer system may, however, include any suitable computer system and associated equipment and may be configured in any suitable manner. In one embodiment, the computer system comprises a stand-alone system. In another embodiment, the computer system is part of a network of computers including a server and a database.

The software required for receiving, processing, and analyzing information may be implemented in a single device or implemented in a plurality of devices. The software may be accessible via a network such that storage and processing of information takes place remotely with respect to users. The system according to various aspects of the present invention and its various elements provide functions and operations to facilitate detection and/or analysis, such as data gathering, processing, analysis, reporting and/or diagnosis. For example, in the present aspect, the computer system executes the computer program, which may receive, store, search, analyze, and report information relating to the human genome or region thereof. The computer program may comprise multiple modules performing various functions or operations, such as a processing module for processing raw data and generating supplemental data and an analysis module for analyzing raw data and supplemental data to generate quantitative assessments of a disease status model and/or diagnosis information.

The procedures performed by the system may comprise any suitable processes to facilitate analysis and/or cancer diagnosis. In one embodiment, the system is configured to establish a disease status model and/or determine disease status in a patient. Determining or identifying disease status may include generating any useful information regarding the condition of the patient relative to the disease, such as performing a diagnosis, providing information helpful to a diagnosis, assessing the stage or progress of a disease, identifying a condition that may indicate a susceptibility to the disease, identify whether further tests may be recommended, predicting and/or assessing the efficacy of one or more treatment programs, or otherwise assessing the disease status, likelihood of disease, or other health aspect of the patient.

The following example is provided to further illustrate the advantages and features of the present invention, but it is not intended to limit the scope of the invention. While this example is typical of those that might be used, other procedures, methodologies, or techniques known to those skilled in the art may alternatively be used.

### Example 1

Use of DELFI Circulating cfDNA-Based Approach to Cancer Detection for Diagnostic Evaluation of Patients with Symptoms of Lung Cancer

Exemplary results are presented, demonstrating that these methods can distinguish lung cancer from noncancer in an elevated-risk cohort presenting with well-established signs and symptoms suggestive of lung cancer.

Participants were adults referred to the Department of Respiratory Medicine at Bispebjerg Hospital in Copenhagen, Denmark (LUCAS cohort) with positive imaging findings on chest radiography or chest CT scan. Mathios, D., et al., Detection and characterization of lung cancer using cell-free DNA fragmentomes. Nat Commun. 2021 Aug. 20; 12(1):5060. Information about symptom(s) at presentation were extracted from reasons for referral documented in medical records. Individuals were excluded who had a cancer diagnosis or known active disease, who were under treatment at the time of enrollment, or whose cancer was determined to have been a metastasis of non-lung origin. The study spanned the period from September 2012 to March 2013, and participant follow-up continued until death or April 2020, whichever occurred first. All participants gave written informed consent. The study was performed according to the Declaration of Helsinki and was approved by the Danish Regional Ethics Committee and the Danish Data Protection Agency.

### Study Design

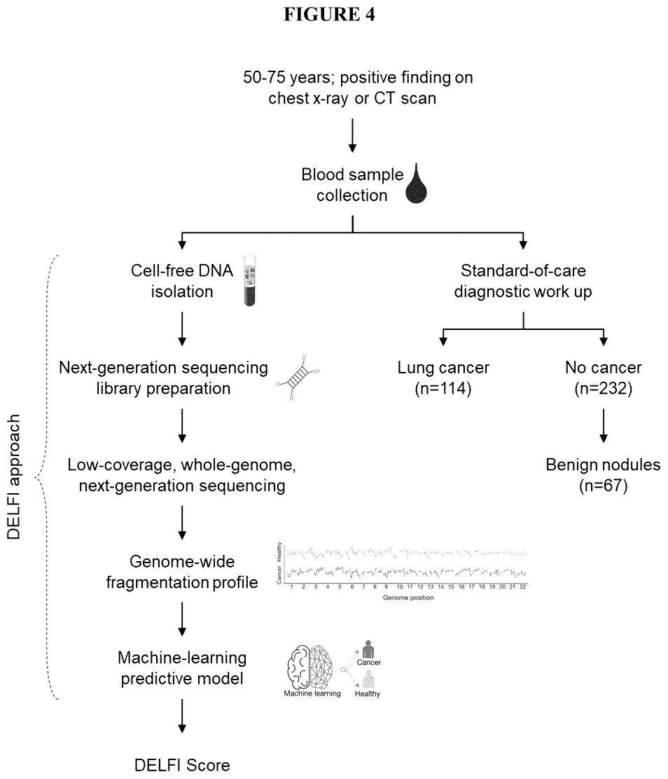

Blood samples were taken from each participant at the initial clinic visit before a diagnosis was made. Samples were processed within 2 hours of collection and stored at −80° C. before cfDNA analyses by DELFI approach, as previously described.[Mathios Nat Commun 2021] Briefly, cfDNA was isolated from 2-4 mL of plasma and stored at −80° C. Next-generation sequencing genomic libraries were prepared in batches from the cfDNA of individuals with lung cancer and no cancer. Libraries were sequenced using 100-bp paired-end runs on the Illumina HiSeq2500 platform (Illumina, Inc., San Diego, CA) at 1-2× coverage per genome. A machine-learning predictive model was used to detect cancer based on compositions of large-scale epigenetic signals in sequenced cfDNA fragments across 2.4 GB of the autosomal genome in patients with lung cancer and in noncancer controls (FIG. 4).

### Statistical Analyses

Demographic and clinical characteristics were described by cancer status. The X² test was used to compare categorical variables and Student's t-test was used to compare continuous variables. Median DELFI scores were reported by lung cancer outcome, number of symptoms reported, and individual symptoms reported, and the distribution of DELFI scores was visualized by the same subgroups. The Kruskall Wallis X² test was used to compare distributions of DELFI scores. Receiver operating curves were constructed from logistic regression models assessing the association between individual reported symptoms (with and without DELFI score) and lung cancer outcome. Area under the curve and 95% confidence intervals were estimated for each receiver operating curve.

### Results

The LUCAS cohort comprised 365 participants, of whom 346 individuals were included in these analyses: 114 with lung cancer and 232 without cancer (FIG. 4). The remaining 19 patients were excluded from the current analyses because they had lung metastases from primary tumors originating from tissues other than lung. Demographic and clinical characteristics of the LUCAS cohort are shown in Table 4. Both the lung cancer and noncancer cohorts had signs and symptoms before diagnosis that are often associated with lung cancer, and the two groups had balanced distribution of the number of signs and symptoms.

The median DELFI score was significantly higher for the group with lung cancer than the group without cancer (0.871 vs 0.203; p<0.001). Among 232 individuals without cancer, 67 were confirmed to have benign lung nodules after an abnormal chest imaging result. The median DELFI scores were similar in noncancer individuals who did and did not have benign nodules (0.208 vs 0.198, p=0.299).

For each symptom, the median DELFI score was significantly higher for individuals with lung cancer who had that symptom than individuals without cancer who had that symptom (FIG. 4; Table 1).

TABLE 1
Median DELFI scores by cancer status and specific
symptom documented.

| | Median DELFI Score | |
|---|---|---|
| Symptom Documented | No cancer | Lung cancer |
| Chest pain | 0.146 | 1.000 |
| Cough | 0.179 | 0.965 |
| Dyspnea | 0.253 | 0.926 |
| Fatigue | 0.181 | 0.976 |
| Hemoptysis | 0.216 | 0.999 |
| Weight loss | 0.208 | 1.000 |

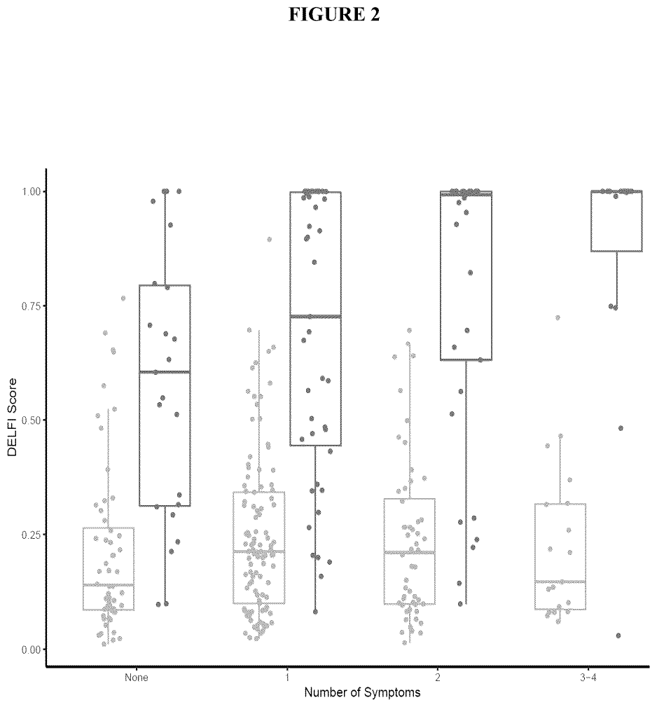

The median DELFI score ranged from 0.926 to 1.000 in the group with cancer and from 0.146 to 0.253 in the group without cancer, depending on the symptom. When the cohort was stratified by number of symptoms, the median DELFI score was significantly higher among patients with lung cancer than without cancer across each stratum of symptom number (FIG. 2; Table 2). The median DELFI score ranged from 0.604 to 1.000 in the group with cancer and from 0.140 to 0.212 in the group without cancer, depending on the number of symptoms.

TABLE 2
Median DELFI scores by cancer status and number of symptoms
documented.

| No. of Symptoms | Median DELFI Score | |
|---|---|---|
| Documented | No cancer | Lung cancer |
| None | 0.140 | 0.604 |
| 1 | 0.212 | 0.727 |
| 2 | 0.211 | 0.993 |
| 3-4 | 0.147 | 1.000 |

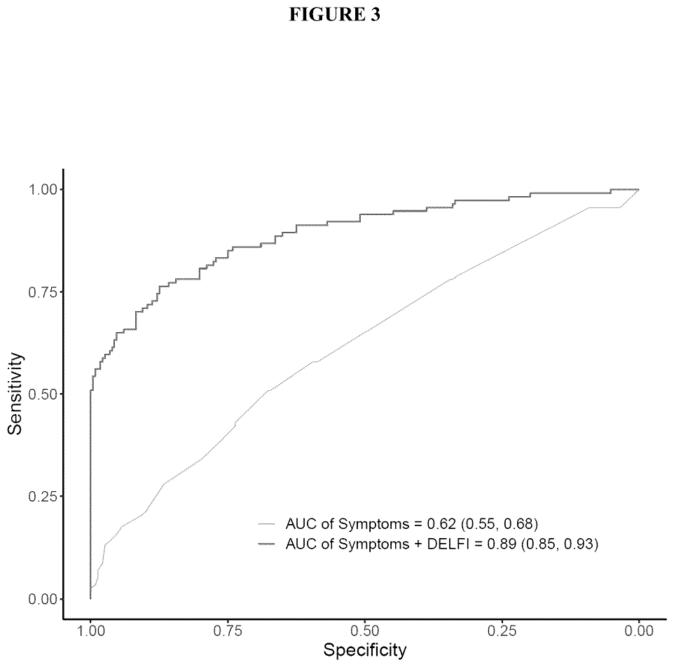

In a logistic regression model of combined symptoms to predict the presence of lung cancer, the area under the curve was 0.62 (95% confidence interval, 0.55 to 0.68; FIG. 3). Adding the DELFI score to a model with combined symptoms improved the area under the curve to 0.89 (95% confidence interval, 0.85 to 0.93; FIG. 3).

### DISCUSSION

Here we demonstrate the potential value of the DELFI approach to detect lung cancer in a cohort that presented for evaluation with signs or symptoms suggestive of lung cancer. By analyzing the fragmentation patterns of cfDNA present in blood plasma, the DELFI platform distinguished lung cancer from noncancer in relatively high-risk individuals, i.e., those with positive imaging results and a variety of suspicious symptoms. Our findings provide proof-of-concept evidence that use of the DELFI approach in the diagnostic workup of individuals with signs and symptoms could shorten the time to diagnostic resolution. Even among a group with high-risk features, the DELFI score could further classify those with relatively higher and lower likelihoods of lung cancer. Those with a higher likelihood of lung cancer could be directed more urgently to undergo LDCT screening and subsequent biopsy. Additional investigations of the performance (e.g., specificity, sensitivity, positive and negative predictive values) of the DELFI approach would be needed to define the clinical utility of a DELFI-based test for such decision-making.

The DELFI approach correctly classified benign lung nodules as noncancer. Lung nodules are common incidental findings on chest imaging. Although frequently benign, lung nodules require additional workup such as imaging scans or biopsy, all of which carry a risk of harm to the patient. The classification of nodules as benign by the DELFI score has the potential to reduce the number of unnecessary procedures that many would otherwise endure.

Our study showed the feasibility of applying the DELFI approach to archived blood specimens that were systematically collected and processed from individuals cared for in routine practice.

In summary, genome-wide interrogation of fragmentation patterns of cfDNA by the DELFI approach represents a novel, noninvasive, feasible, and potentially useful tool to aid the diagnosis of lung cancer in individuals with suggestive signs and symptoms. By identifying individuals who are more (and less) likely to have lung cancer, use of the DELFI approach in these ambiguous scenarios could allow more informed and timely referrals for additional imaging studies or biopsies. This approach could reduce the number of unnecessary procedures and shorten the time to diagnostic resolution for many. Further investigations of the DELFI approach are needed.

TABLE 3
Summary of guidelines for the diagnostic workup of individuals who
present with symptoms suspicious of lung cancer

| Guideline | |
|---|---|
| Group | Recommendations |
| | Indications for chest CT scan and/or referral to a specialist: |
| CCO Lung | Chest x-ray findings suspicious of lung cancer OR |
| Cancer | High suspicion of lung cancer based on clinical |
| Referral | judgment despite normal chest x-ray findings |
| Working | |
| Group | |
| NICE | Chest x-ray findings suggest lung cancer OR |
| | ≥40 years of age with unexplained hemoptysis |
| NZGG | Chest x-ray findings suggest lung cancer OR |
| | High suspicion of lung cancer despite normal |
| | chest x-ray findings OR |
| | ≥40 years of age, ever-smoker, with |
| | unexplained hemoptysis |
| | Indications for an urgent chest x-ray if any of the following: |
| CCO Lung | Within 2 working days if any of the following: |
| Cancer | Dysphagia |
| Referral | Finger clubbing |
| Working | Hemoptysis |
| Group | Lymphadenopathy (suspicious) |
| | Features suggestive of cancer metastasis to or from the lung |
| | Feature suggestive of paraneoplastic syndromes |
| NICE | If ≥40 years of age with any of these symptoms: |
| | Chest infection (persistent or recurrent) |
| | Chest signs consistent with lung cancer |
| | Finger clubbing |
| | Supraclavicular lymphadenopathy or persistent cervical |
| | lymphadenopathy |
| | Thrombocytosis |
| NZGG | Hemoptysis (unexplained) |
| | Indications for a chest x-ray: |
| CCO Lung | Within 2 weeks (or sooner if known risk |
| Cancer | factors for lung cancer): |
| Referral | Appetite loss or weight loss |
| Working | Chest or shoulder pain |
| Group | Chest signs |
| | Cough |
| | Hoarseness |
| | Shortness of breath |
| NICE | Within 2 weeks if ≥40 years of age |
| | with ≥2 unexplained symptoms, or |
| | if ever-smoked and ≥1 unexplained symptoms: |
| | Appetite loss or weight loss |
| | Chest pain |
| | Cough |
| | Fatigue |
| | Shortness of breath |
| NZGG | If any of the following unexplained |
| | signs/symptom lasting >3 weeks |
| | (or less if known risk factors for lung cancer): |
| | Appetite loss or weight loss |
| | Chest or shoulder pain |
| | Chest signs |
| | Cough |
| | Fatigue |
| | Finger clubbing |
| | Hoarseness |
| | Shortness of breath |
| | Supraclavicular lymphadenopathy or cervical |
| | lymphadenopathy |
| | Features suggestive of metastasis from lung cancer |

CCO = Cancer Care Ontario; NICE = National institute for Health and Care Excellence; NZGG = New Zealand Guidelines Group

TABLE 4
Participant demographics and clinical characteristics

| | | No cancer | Lung cancer | P |
|---|---|---|---|---|
| Characteristic | | (n = 232) | (n = 114) | value |
| Age, y | Mean (SD) | 62.3 (14.2) | 69.7 (9.4) | <. 001 |
| | Median range) | 64.0 (19.0-96.0) | 69.5 (44.0-91.0) | |
| Sex, n (%) | Female | 110 (47.4%) | 61 (53.5%) | .341 |
| | Male | 122 (52.6%) | 53 (46.5%) | |
| No. of | None | 56 (24.1%) | 23 (20.2%) | .188 |
| symptoms | 1 | 105 (45.3%) | 43 (37.7%) | |
| documented, | 2 | 52 (22.4%) | 33 (28.9%) | |
| n (%) | 3-5 | 19 (8.2%) | 15 (13.2%) | |
| Type of s | Chest pain | 32 (13.8%) | 23 (20.2%) | .171 |
| ymptom, n | Cough | 115 (49.6%) | 55 (48.2%) | .907 |
| (%)^{a} | Dyspnea | 52 (22.4%) | 32 (28.1%) | .308 |
| | Fatigue | 3 (1.3%) | 8 (7.0%) | .012 |
| | Hemoptysis | 27 (11.6%) | 8 (7.0%) | .250 |
| | Weight loss | 40 (17.2%) | 33 (28.9%) | .018 |
| Smoking | Current, n (%) | 71 (30.6%) | 47 (41.2%) | <.001 |
| status | Former, n (%) | 117 (50.4% | 61 (53.5%) | |
| | Never, n (%) | 44 (19.0%) | 3 (2.6%) | |
| | Missing, n (%) | 0 (0%) | 3 (2.6%) | |
| Pack-years | Mean (SD) | 26.5 (22.8) | 44.6 (26.2) | <.001 |
| | Median (range) | 25.0 (0-110) | 40.0 (0-150) | |
| Cancer | I | NA | 15 (13.2%) | NA |
| stage, n (%) | II | NA | 7 (6.1%) | |
| | III | NA | 35 (30.7%) | |
| | IV | NA | 57 (50.0%) | |
| DELFI | Median (range) | 0.203 (0.011- | 0.871 | <.001 |
| test score | | 0.896) | (0.030-1.00) | |

ªIndividuals with more than one symptom documented are counted more than once (once for each symptom that was documented); DELFI = DNA evaluation of fragments for early interception; NA = not applicable; SD = standard deviation.

Although the invention has been described with reference to the presently preferred embodiment, it should be understood that various modifications can be made without departing from the spirit of the invention.

1. A method comprising:
processing cfDNA fragments from a sample obtained from a subject and generating sequencing libraries;
subjecting the sequencing libraries to whole genome sequencing to obtain sequenced fragments, wherein genome coverage is about 9× to 0.1×;
mapping the sequenced fragments to a genome to obtain genomic intervals of mapped sequences;
analyzing the genomic intervals of mapped sequences to determine cfDNA fragment lengths and amounts to establish a composite cfDNA fragmentation profile using the cfDNA fragment lengths and amounts;
analyzing one or more demographic or clinical characteristics from the subject which are associated with a type of cancer to be identified; and
detecting a composite cfDNA fragmentation profile based on lengths and amounts that is variable relative to a reference cfDNA fragmentation profile from a healthy subject,
wherein increased variability of the cfDNA fragmentation profile and the presence of one or more demographic or clinical characteristics indicate that the subject has the type of cancer.

2. The method of claim 1, wherein the genomic intervals are non-overlapping.

3. The method of claim 1, wherein the genomic intervals each comprise thousands to millions of base pairs.

4. The method of claim 1, wherein a cfDNA fragmentation profile is determined within each genomic intervals.

5. The method of claim 1, wherein the cfDNA fragmentation profile comprises a median fragment size.

6. The method of claim 1, wherein the cfDNA fragmentation profile comprises a fragment size distribution.

7. The method of claim 1, wherein the cancer is selected from the group consisting of head and neck cancer, lung cancer, breast cancer, esophageal cancer, gastric cancer, bile duct cancer, liver cancer, pancreatic cancer, colorectal cancer, kidney cancer, bladder cancer, ovarian cancer, and endometrial cancer.

8. The method of claim 7, wherein the type of cancer is lung cancer.

9. The method of claim 7, wherein one or more clinical characteristic is selected from the group consisting of pain, involuntary weight loss, fever, fatigue, skin changes, dyspnea, cough, hoarseness, dysphagia, unusual bleeding, anemia, change in intestinal or urinary habits, or swelling or lumps anywhere in the subject's body.

10. The method of claim 7, wherein one or more demographic characteristic is selected from the group consisting of age, sex and smoking status.

11. The method claim 1, further comprising administering to the subject identified as having the type of cancer, a therapeutic agent suitable for the treatment of the type of cancer.
